(19) United States
(12) Patent Application Publication
(10) Pub. No.: US 2008/0171097 A1
(43) Pub. Date: Jul. 17, 2008

(54) PRODUCT LIQUORICE ROOT AND OAK BARK IN COMBINATION WITH DEXAPANTHENOL

(76) Inventor: Gundula GESKE, Owen (DE)

Correspondence Address: MORRIS MANNING MARTIN LLP, 3343 PEACHTREE ROAD, NE, 1600 ATLANTA FINANCIAL CENTER, ATLANTA, GA, 30326, US

(21) Appl. No.: 12/013,770

(22) Filed: Jan. 14, 2008

(30) Foreign Application Priority Data
Jan. 15, 2007 (DE) 10 2007 002 170.6

(51) Int. Cl. A61K 36/236; A61K 36/49; A61K 31/16; A61P 31/00

(52) U.S. Cl. 424/757; 424/771; 514/629

(57) ABSTRACT

The invention relates to a pharmaceutical composition and the use of same for treating diseases that are caused by microorganisms, and as a cosmetic. In one embodiment, the pharmaceutical composition comprises an extract of liquorice root and oak bark in combination with dexpanthenol.

### FIELD OF THE INVENTION

The invention relates to a pharmaceutical composition, and more particularly to a pharmaceutical composition comprising an extract of liquorice root and oak bark in combination with dexpanthenol, and the use of same for treating diseases that are caused by microorganisms, and as a cosmetic.

### BACKGROUND OF THE INVENTION

Inflammations in the lips and in the pharyngeal space are a very widespread problem. These inflammations are often due to viral infections. So-called herpes viruses are often the cause of such infections. There are many different types of these viruses but only a few of them cause a disease in humans.

Although these various herpes viruses behave differently, they all have one unpleasant property: once they have penetrated the body and caused a primary infection, they do not leave the body again. Rather they retreat along the nerve fibres into the ganglia as dormant “latent” viruses. Under certain circumstances these viruses can then become active again and cause a clinical picture.

A herpes infection is a particularly widespread, unpleasant problem which manifests itself in the form of blisters on the lips, the so-called herpes labialis. This is the most frequent form of a herpes disease. The infection is due to the herpes simplex virus type 1. About 30% to about 70% of the population are already infected with this virus in childhood. However, symptoms do not necessarily occur in the infected persons. After the primary infection, the viruses settle in the nerve ganglia and in many affected persons cause very unpleasant so-called herpes blisters from time to time which mainly occur on the lips. These so-called relapses primarily appear under certain circumstances, for example, under stress, extreme exposure to the sun, skin irritation or other stresses. It is first felt as itching and a feeling of tension. Then grouped blisters on a reddened background occur which later dry to form crusts and heal.

Additionally, herpes viruses can also cause quite different clinical pictures. For example the children's disease chicken pox is also caused by herpes viruses. The viruses remain as latent viruses in the body and can become active again many years later and cause so-called shingles, the herpes zoster. Furthermore, herpes infections can also manifest themselves in the eye or as herpes of the genitals or viral meningitis.

There are an abundance of home remedies that are recommended for herpes diseases and in particular herpes labialis. However, these remedies are in general not very effective. To a certain degree the active substance Aciclovir has proven to be effective in the treatment of herpes blisters on the lips. However, treatment with Aciclovir is in any case purely symptomatic so that relapses and thus a reactivation of the latent viruses occur again and again.

In view of a continuously increasing need to provide alternatives to synthetic products having the same or a similar spectrum of action for the treatment of diseases that are caused by microorganisms and in particular viruses, various products were described in the past years which are based on naturally occurring products.

The Japanese Patent Application JP 07179354 discloses an antiviral agent which contains an aqueous extract of a mixture of ten different plant starting materials including liquorice root (Glycyrrhiza glabra) and oak bark (Quercus spec.).

EP 0 568 001 A2 describes an agent comprising at least one aqueous or methanolic extract of a crude drug such as for example liquorice root and oak bark for treating viral diseases that are caused among others by cytomegaloviruses.

European Patent Application EP 1 238 672 A1 describes a pharmaceutical combination preparation which contains an aqueous ethanolic extract of liquorice root and oak bark and can be used to treat viral diseases such as herpes labialis.

However, the previously mentioned compositions have a number of disadvantages. JP 07179354 and EP 0 568 001 A2 each disclose an extraction of crude drugs in boiling solvents. The preparation of the extract of liquorice root and oak bark according to EP 1 238 672 A1 takes place at temperatures of 10° C. to 80° C., where room temperature and a range of 45° C. to 55° C. are regarded to be equally preferred.

In view of the fact that many natural substances do not tolerate the above-mentioned high temperatures and either decompose or evaporate, numerous active substances are inevitably completely removed from an extract prepared in this manner and thus the spectrum of action of such an extract differs considerably from an extract prepared at lower temperatures or under milder extraction conditions.

Furthermore, the combination preparation disclosed in EP 1 238 672 A1 has deficits with regard to tolerance. Thus, according to the above-mentioned patent application, an aqueous ethanolic extract is prepared, which is used as a pharmaceutical preparation without addition of further substances. However, the high ethanol content of the combination preparation results in a rapid drying out of the mucous membranes which is generally undesired for the applications that are described in EP 1 238 672 A1.

Therefore, a heretofore unaddressed need exists in the art to address the aforementioned deficiencies and inadequacies.

### DETAILED DESCRIPTION OF THE INVENTION

An object of the present invention is to provide a product that at least partially solves the problems associated with the compositions described in the prior art. This object is achieved according to the invention by a pharmaceutical composition comprising an extract of liquorice root and oak bark in combination with dexpanthenol which is administered in a suitable form.

In a first aspect, the present invention relates to a pharmaceutical composition comprising an extract of liquorice root and oak bark in combination with dexpanthenol.

Surprisingly, it was found in the present invention that a composition that contains dexpanthenol in addition to an extract of liquorice root and oak bark is particularly suitable for treating diseases that are caused by microorganisms. It was found that synergistic effects of the above-mentioned components result in a considerably improved effect or tolerance of the composition according to the invention as compared to the preparations disclosed in the prior art.

The term “extract of liquorice root and oak bark” as used within the present application refers to an extract which is obtained by extracting a mixture of liquorice root and oak bark as well as an extract which represents a mixture of an extract of liquorice root and an extract of oak bark. Although both variants of the extraction are possible and are encompassed by the present invention, a common extraction of the two components liquorice root and oak bark is preferred.

A first component of the pharmaceutical composition according to the invention is liquorice root. The dried roots of the liquorice plant (Glycyrrhiza glabra) are used as liquorice root. This plant is a member of the Fabaceae family and occurs especially as a wild-growing herbaceous plant in Southern Europe to West Siberia, South America, the Near East and Central Asia as well as in Australia. In addition liquorice is also grown on warm sandy soils especially in Eastern Europe. The plant is woody and can grow up to a height of 2 m. In order to harvest the roots, the up to 2 cm thick side roots are cut off and dried after 3 to 4 years. The harvest can take place every three years from late autumn to spring. The drug has a hardly discernible smell and tastes extremely sweet. The roots and the juice of the liquorice plant have already been used for a long time in medicine. For example the dried roots are used as a tea mixture. A concentrated extract of liquorice root is claimed to be helpful for the treatment of gastric ulcers. Thickened liquorice juice is now used widely as an important component of liquorice products.

The dried bark of young trunks and branches of the pedunculate or summer oak (Quercus robur) and of the sessile or winter oak (Quercus petraea) are used as oak bark which represents the second component of the composition according to the invention. Oak bark can be obtained above all from Eastern and Southeastern European countries where it is cultivated. The bark is usually harvested from March to April i.e. before the leaves develop, when the trees are about 3 to about 10 years old. Oak bark has been used for a long time as a medicinally effective drug especially due to its high tannin content. The resulting astringent action makes it suitable as a bath additive for alleviating inflammatory skin diseases and as an agent for treating diarrhoeal diseases.

Dexpanthenol which is metabolized in the body of mammals to pantothenic acid (vitamin B5), is used as the third component of the pharmaceutical composition according to the invention. Pantothenic acid, in turn, is involved in numerous important metabolic processes as a component of coenzyme A and is crucial for the maintenance and regeneration of tissue.

In accordance with the invention, the compositions disclosed in the present application comprise the combination described above of extract and dexpanthenol in an amount from about 1 to about 100%, more preferably from about 50 to about 100% and most preferably from about 90 to about 100% based on the total weight of the pharmaceutical composition. According to the invention, the weight ratio of extract to dexpanthenol can be between about 99:1 and about 90:10, a range between about 98:2 and about 95:5 being preferred.

The extract of liquorice root and oak bark is advantageously obtained with the aid of extraction agents that are commonly used to prepare plant extracts. The use of extraction pairs such as for example a solvent mixture of water and an organic solvent is preferred. In a preferred embodiment, the organic solvent is an aliphatic alcohol having 1 to 6 carbon atoms, more preferably an aliphatic alcohol having 2 to 4 carbon atoms, and most preferably ethanol. However, for example oil extracts or pressed juices of the plant drugs are also encompassed by the invention. Extraction agents which extract lipophilic and hydrophilic components of the plant drugs are particularly preferred.

A solvent mixture of water and ethanol in which the volume percent of ethanol is adjusted to about 20% to about 80% has proven to be an extremely advantageous extraction agent within the present invention. The volume percent of ethanol is more preferably about 40% to about 70%, most preferably from about 45% to about 55%. The water used to prepare the solvent mixture can be deionized water but also normal tap water.

The plant drugs are extracted according to the invention in a temperature range of about 15° C. to about 35° C. Temperatures of about 20 to about 30° C. are preferred, and a temperature of about 25° C. (room temperature) is most preferred. Under these conditions, an adequate extraction of the plant ingredients takes place under very practicable and mild conditions.

In a preferred embodiment of the invention, the extraction is carried out statically i.e. the preparation is not moved during the extraction with a suitable extraction agent. However, it is indeed possible and may under certain circumstances be advantageous to move, e.g. stir or shake, the preparation during extraction.

The extraction is advantageously carried out for several hours. Preferred times are about 2 hours to about 48 hours. The duration of the extraction period may to a certain extent depend on the selected extraction temperature. Thus, it can be advantageous to carry out an extraction at lower temperatures for a longer period. For example an extraction at room temperature for 24 hours and an extraction at a temperature of 40° C. for 8 hours can be advantageous. However, adequate amounts of the ingredients of plant drugs are also extracted over shorter extraction times and also longer extraction times are generally not disadvantageous.

About 1 to about 5 parts liquorice root and about 1 to about 3 parts oak bark are used for the extract of liquorice root and oak bark. These parts refer to the weights of the plant drugs. Hence, the weight ratio of liquorice root to oak bark can according to the invention be from about 5:1 to about 1:3 for the preparation of the extract. In a particularly preferred embodiment of the invention, the weight ratio of liquorice root to oak bark is 2:1. This ratio of the two drugs has proven to be particularly advantageous with regard to the effect.

The usual forms of preparation of the drugs can be used to produce the extract. Liquorice root and oak bark are advantageously used in a dried and/or cut form. These so-called cut drugs are preferably used in a size of about lentils or beans. This has the advantage that after the extraction the plant components readily sediment in the preparation and the extract can be simply poured off or coarsely filtered. It is, however, also possible to use the drugs as a powder i.e. as ground plant parts. In this case a filtration is usually necessary for further processing after the extraction.

The extract obtained after the extraction can be processed further in various ways. For example the extract can be concentrated in order to thus achieve a higher concentration of active substance. However, the concentration of active substance in the extract is usually so high after extraction that a further concentration is unnecessary and, after combination with dexpanthenol and optionally other additives etc., the extract can be used in a liquid form by a consumer or can be administered to a patient without further processing. This is particularly advantageous because the production of the composition in this manner is exceedingly simple and cheap. Hence, the liquid extract can be applied as such as a tincture on the lips or in the pharyngeal space.

It is additionally possible to provide the pharmaceutical composition according to the invention at least partially in a dried form. For this purpose, the extraction agent is removed after extraction by evaporation, e.g. by freeze drying, so that the extracted ingredients of the drugs are present in a dried form. These ingredients can be used further or processed further in various ways.

According to the invention, the pharmaceutical composition contains the extract of liquorice root and oak bark in combination with dexpanthenol preferably together with one or more pharmaceutically acceptable carriers, humectants and/or additives, the term “pharmaceutically acceptable carrier” denoting one or more liquid, semi-solid or solid diluents, fillers or other substances which are suitable for an administration to mammals including humans.

The term “pharmaceutically acceptable” as used herein refers to any non-toxic material which does not influence the effectiveness of the biological activity of the active substance. Such materials can comprise pharmaceutically acceptable concentrations of salts, buffers, preservatives or such like, wherein in the case of medical applications the salts should be pharmaceutically acceptable salts. Non-pharmaceutically acceptable salts could be used provided pharmaceutically acceptable salts can be prepared from such salts.

The term “carrier” in the sense of the present invention refers to any organic or inorganic, natural or synthetic substance which can be combined with the active substance to simplify administration. Examples of such carriers comprise, but are not limited to, organic or inorganic solvents, starch, lactose, mannitol, methyl-cellulose, talcum, gelatin, agar-agar, calcium phosphate, magnesium stearate, animal and plant fats, higher-molecular fatty acids or higher-molecular polymers.

In a preferred embodiment, the pharmaceutically acceptable carrier serves as a foodstuff, semiluxury food and/or personal hygiene product. Thus, the combination of extract and dexpanthenol can for example be incorporated into a chewing sweet, into chewing gum or also into toothpaste. In this manner, the effective ingredients of the composition according to the invention reach the site of disease by chewing or sucking or cleaning the teeth and can display their action at this site. Incorporation into a chewing gum for example has the advantage that such a chewing gum remains in the oral cavity or pharyngeal space for a relatively long period and thus the active ingredients of the composition can be effective there for a long period.

According to the invention, the pharmaceutical composition of the present application additionally comprises one or more suitable humectants. Preferred examples of humectants are pharmaceutically tolerated polyalcohols such as propylene glycol, in particular 1,2-propylene glycol, pentylene glycol, in particular 1,2-pentanediol, glycerol and/or polyethylene glycol.

Additives in the sense of the present invention for example comprise reagents for adjusting the pH, buffers, diluents, processing aids such as emulsifiers, preservatives, stabilizers, antioxidants, light protecting agents, dyes and suchlike. In particular compounds of alkali or alkaline earth metals which undergo an alkaline reaction such as hydroxides, hydrogen carbonates, carbonates etc. which among others also include organometallic compounds are preferably used as reagents for adjusting the pH. It is equally possible to optionally also use acids including organic and inorganic acids to adjust the pH. Suitable buffers comprise, but are not limited to, acetic acid, citric acid, tartaric acid, boric acid or phosphoric acid in combination with their corresponding bases. Preservatives which can be used in the present invention comprise, but are not limited to, benzalkonium chloride, chlorobutanol, thiomersal or parabenes.

The pharmaceutical compositions according to the invention are preferably formulated as a spray, gel, cream, ointment, lotion, tablet or suppository. However, the compositions can also be administered in the form of aerosols, aqueous or non-aqueous solutions, foams, emulsions, suspensions or other suitable formulations.

The compositions according to the invention are preferably administered topically. This topical administration is especially suitable for treating herpes blisters or localizable inflammations in the pharyngeal space. In this connection, they can be applied using a finger or other aids. Application with the aid of an applicator is particularly preferred because this avoids contaminating the pharmaceutical composition. For example the composition can be applied with a cellulose pad and in particular with a conventional cotton bud. However, other applicators are also indeed possible. In this connection, it is particularly advantageous when the applicator has a certain reservoir of liquid.

Other forms of administration which are suitable for the application of compositions in the sense of the present invention are known to a person skilled in the field of pharmacology or medicine and comprise, but are not limited to, subcutaneous, intradermal, transdermal, oral, nasal, inhalative, rectal or intravenous administration.

The compositions according to the invention are administered such that per administered dose preferably an amount in the range of about 10 mg to about 10 g and in particular an amount in the range of between about 100 mg to about 1 g of the combination of extract and dexpanthenol in a dry form is provided. In a preferred embodiment, the administered amount is a pharmaceutically effective amount.

According to the invention, the compositions described in the present application are used to treat diseases that are caused by microorganisms. The term “treatment” as used herein refers to a therapeutic treatment in which the recipient is administered the compositions according to the invention in an amount effective for prevention, alleviation or elimination of the disease or disorder.

The term “microorganisms” in the sense of the present invention comprises bacteria, fungi, yeasts, protozoa, algae and viruses. The microorganisms which cause the disease are preferably bacteria, fungi or viruses and most preferably viruses.

As will be shown on the basis of the example of the present invention, the composition according to the invention has an extremely surprising positive effect in the treatment of viral diseases in particular. The observed synergistic effect of the components of the pharmaceutical composition according to the invention is presumably based on a mutual promotion of virologic and immunologic effects of the various ingredients of liquorice root and oak bark as well as dexpanthenol.

In the case of a viral infection, the viruses interact with surface receptors of cells whereupon the viruses can penetrate into the cells. The viruses multiply within the cells at the cell's expense and subsequently leave the cell whereupon the cell is destroyed. One of the effects of the pharmaceutical composition according to the invention is presumably that the surface receptors are damaged which prevents attachment of the viruses and in this manner the viruses cannot penetrate into the cell. This damage of surface receptors is presumably an unspecific interaction of various ingredients of the composition according to the invention with the surface proteins so that the composition according to the invention displays a relatively wide variety of activities. In addition, the composition according to the invention presumably also has an effect on the multiplication processes of the viruses within the cell.

In addition, the composition according to the invention presumably induces a large number of immunological defense mechanisms. These for example include the activation of antigen-specific T lymphocytes by ingredients of the composition according to the invention. These activated T lymphocytes directly attack the virus-infected cells. Furthermore, interferon gamma (IFNγ), a cytokine important for the development and maintenance of inflammatory processes, is released by these activated T lymphocytes. This messenger amplifies the activation of T lymphocytes and additionally also activates the natural killer cells. These cells, in turn, also attack the virus-infected cells. In addition, interferon gamma can have a direct inhibitory effect on virus multiplication within the infected cells. Overall, the composition according to the invention has an activating effect on the immune system and starts up numerous mechanisms which combat virus multiplication and the virus-infected cells.

In addition to the acute anti-viral effect, the composition according to the invention also acts against a renewed activation of the latent viruses. This particularly advantageous effect of the composition according to the invention is presumably also due to the mutually supporting antiviral and immunological effects of the composition. These synergistic effects are presumably the reason for the surprising therapeutic successes of the composition according to the invention.

Due to the very different points of attack of the composition which mutually support each other, the development of resistances by the respective pathogens is additionally advantageously avoided. Such developing resistances are a very major problem especially in the everyday hospital routine. This applies especially to chemically defined preparations which can be initially very effective but later do not develop any effect at all because the corresponding pathogens no longer respond to this preparation due to mutations or suchlike. When using the composition according to the invention, the development of such resistances is advantageously ruled out due to presumably the very different mechanisms of action.

Diseases which can be treated with the aid of the pharmaceutical composition according to the invention comprise any diseases caused by microorganisms but especially viral diseases. Diseases of the skin and/or mucous membrane, in particular of the mucous membrane of the mouth or nose, can be preferably treated using the pharmaceutical composition of the present invention. The pharmaceutical composition is more preferably used to treat diseases whose symptoms manifest themselves in the area of the lips and/or pharyngeal space, the treatment of diseases that are caused by herpes viruses and in particular herpes labialis being most preferred. However, the diseases which can be treated with the composition according to the invention are not at all limited to herpes diseases. Thus, other diseases which are due to viral or bacterial infections, and in particular common colds or influenza infections, can be treated very effectively with the pharmaceutical composition of the present invention.

A second aspect of the present invention concerns the use of liquorice root and oak bark in combination with dexpanthenol for the preparation of a pharmaceutical composition for treating diseases that are caused by microorganisms and in particular viruses. Reference is made to the above-mentioned details with regard to this use.

The present invention additionally concerns the use of an extract of liquorice root and oak bark in combination with dexpanthenol as a cosmetic. The cosmetic is used to protect, care for, clean and/or improve the condition of the skin and/or of mucous membranes, an application of the cosmetic being advantageous especially following a viral or bacterial infection.

The present invention is further elucidated by the following example.

### EXAMPLE

In order to prepare an extract, 6.67 g dried liquorice root which is cut into small pieces and 3.33 g dried oak bark cut into small pieces is weighed in and placed in a closable container. After pouring 75.94 g ethanol (96%) and 91.69 g deionized water over the drugs, the mixture was stirred for about 5 minutes, the container was subsequently closed and the preparation was allowed to stand for about 72 hours at room temperature. After this period had elapsed, the preparation was filtered and the filtrate was weighed. 7.50 g dexpanthenol (75%) was added to 142.50 g of the filtrate obtained in this manner, it was divided into portions, filled into suitable vessels and used as a pharmaceutical composition.

This composition was applied in the oral and/or pharyngeal cavity of voluntary test persons. When there were signs that herpes blisters were about to occur or when herpes blisters had already occurred, the composition was applied several times daily to the affected skin sites using a cotton bud. In the case of other inflammations in the oral and/or pharyngeal cavity the solution was also either applied locally or a dilution of this solution was gurgled.

There was a considerable alleviation of the ailments in all cases. With regard to a herpes disease in the lip region, the effectiveness of the composition according to the invention was considerably superior with regard to effectiveness and tolerance over the compositions described in the prior art containing liquorice root and oak bark and the effectiveness was at least equal to that of Aciclovir. Moreover, the composition according to the invention had a considerably better long-term action than Aciclovir. After treatment of acute herpes blisters with Aciclovir and healing of these blisters, relapses very often occurred again within the following 14 days in a test subject in the case of conventional treatment. Treatment with the composition according to the invention enabled, on the one hand, a considerable improvement of the acute herpes symptoms and, on the other hand, reoccurrence is prevented. This long-term effect which is presumably due to the synergistic effects of the various ingredients of the composition on the immune system shows the considerable advantages of the composition according to the invention as compared to conventional compositions when treating viral diseases.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

What is claimed is:

1. A pharmaceutical composition comprising an extract of liquorice root and oak bark in combination with dexpanthenol.

2. The pharmaceutical composition of claim 1, characterized in that the weight percentage of the combination of the extract and the dexpanthenol is about 1 to about 100% based on the total weight of the pharmaceutical composition.

3. The pharmaceutical composition of claim 2, characterized in that the weight ratio of the extract to the dexpanthenol is about 99:1 to about 90:10.

4. The pharmaceutical composition of claim 1, characterized in that a mixture of water and an organic solvent is used as an extraction agent for the extract.

5. The pharmaceutical composition of claim 4, characterized in that ethanol is used as the organic solvent.

6. The pharmaceutical composition of claim 5, characterized in that the volume content of the ethanol in the extraction agent is about 20% to about 80%.

7. The pharmaceutical composition of claim 1, characterized in that the extraction is carried out at a temperature of about 15° C. to about 35° C.

8. The pharmaceutical composition of claim 1, characterized in that the weight ratio of the liquorice root to the oak bark in the production of the extract is about 5:1 to about 1:3.

9. The pharmaceutical composition of claim 1, further comprising at least one of pharmaceutically acceptable carriers, humectants and additives.

10. The pharmaceutical composition of claim 9, characterized in that the pharmaceutically acceptable carrier serves as a foodstuff, semiluxury food and/or personal hygiene product.

11. The pharmaceutical composition of claim 9, characterized in that the humectant comprises at least one of propylene glycol, pentylene glycol, glycerol and polyethylene glycol.

12. The pharmaceutical composition of claim 1, characterized in that it is formulated as a spray, gel, cream, ointment, lotion, tablet or suppository.

13. The pharmaceutical composition of claim 1, characterized in that it is formulated for topical administration.

14. The pharmaceutical composition of claim 1, characterized in that it is provided for treating diseases that are caused by microorganisms.

15. The pharmaceutical composition of claim 14, characterized in that the microorganisms are viruses.

16. The pharmaceutical composition of claim 14, characterized in that one of the diseases is a disease of the skin and/or a mucous membrane.

17. The pharmaceutical composition of claims 14, characterized in that the symptoms of the diseases occur in the region of the lips and/or pharyngeal space.

18. The pharmaceutical composition of claim 14, characterized in that one of the diseases is a herpes disease.

19. Use of an extract of liquorice root and oak bark in combination with dexpanthenol for treating diseases that are caused by microorganisms.

20. Use of an extract of liquorice root and oak bark in combination with dexpanthenol for the preparation of a pharmaceutical composition for treating diseases that are caused by microorganisms.

21. A method for treating diseases that are caused by microorganisms, characterized in that a patient is administered with a pharmaceutically effective amount of an extract of liquorice root and oak bark in combination with dexpanthenol.

22. Use of an extract of liquorice root and oak bark in combination with dexpanthenol as a cosmetic.
